(19) United States
(12) Patent Application Publication
(10) Pub. No.: US 2006/0013355 A1
(43) Pub. Date: Jan. 19, 2006

(54) Method for the reconstruction of sectional images from detector measurement data of a tomography device

(76) Inventor: Bjoern Heismann, Erlangen (DE)

Correspondence Address: HARNESS, DICKEY & PIERCE, P.L.C., P.O.BOX 8910, RESTON, VA, 20195, US

(21) Appl. No.: 11/182,006

(22) Filed: Jul. 15, 2005

(30) Foreign Application Priority Data
Jul. 16, 2004 (DE) 10 2004 034 500.7

(51) Int. Cl. H05G 1/60; A61B 6/00; G01N 23/00; G21K 1/12

(52) U.S. Cl. 378/4

(57) ABSTRACT

A method includes dividing a scanning volume into a multiplicity of partial volumes. For each partial volume, a reference beam is sought, which intersects the partial volume and which is at the greatest distance from the system axis. Further, the absorption coefficients of each partial volume are calculated exclusively with absorption values that originate from beams whose distance is greater than or equal to the distance between the reference beam and the system axis.

The present application hereby claims priority under 35 U.S.C. §119 on German patent application number DE 10 2004 034 500.7 filed Jul. 16, 2004, the entire contents of which is hereby incorporated herein by reference.

### FIELD

The invention generally relates to a method for the reconstruction of sectional images from detector measurement data of a tomography device. For example, it relates to a method involving a tomography device having at least one radiation source which is moved about a system axis, and at least one at least single-row detector lying opposite, which measures the absorption of the radiation emerging from the radiation source after penetrating an examination object.

Furthermore, the invention generally relates to a CT device equipped with device/method/way for carrying out reconstructions of CT images.

### BACKGROUND

In principle, two different methods are generally known with regard to the reconstruction of CT images. In this regard, reference is made to the publication “Computertomographic [Computer tomography] Willi A. Kalender, ISBN 3-89578-082-0”. Chapter 1.2.3 illustrates the two variants of the calculation methods. These concern firstly an explicit calculation method in which a cross section of the examination object is divided into a matrix having N×N matrix elements and these N² unknown values of the N×N image matrix are determined by solving a system of linear equations. In the simplest case of a 2×2 image matrix having only four pixels, a system of four equations with four unknowns results from in each case two measurements from two directions, which system can be solved in a straightforward manner.

With a higher resolution and thus with an enlarged matrix, however, the computation times rise to a disproportionate extent. Thus, an implementation in practice for a matrix of the present-day order of magnitude of 512×512 image elements is scarcely feasible. Furthermore, there also arise in this case, in principle, error propagation problems that lead to an insolubility of such systems of linear equations, thus precluding explicit calculation in practice.

In practice, an approximation method is used nowadays instead of explicit calculation of the image values, in which method the image arises from the measured sinograms by convolution and back projection. This method is also supplemented, particularly in spiral CT, by a preceding rebinning, that is to say a reordering of the scanning beams, if appropriate paired with interpolation methods that generate a measurement data series in the respectively desired geometrical form from the measurement data obtained, CT images subsequently being reconstructed by convolution and back projection. These reconstruction methods are also very complicated in part and require enormous computation capacities despite approximate calculation.

### SUMMARY

An object of an embodiment is to find a method for the reconstruction of sectional images from detector measurement data of a tomography device which concerns itself better than the methods of the prior art with the actual geometry of the scanning systems of modern CT devices in which radiation sources emit a fan-type beam toward a detector and the radiation source circulates around the examination object under consideration on an—imaginary—cylinder surface of the examination object.

The inventor has recognized that, in contrast to the hitherto customary division of the examination region into rectangular two-dimensional or three-dimensional matrices, a division of the examination region into concentrically arranged shells with an additional uniform subdivision of each shell into subsegments, given corresponding adaptation of the computational model, the computation can be substantially simplified. As such, an explicit calculation of the adsorption coefficients of the individual shell segments now becomes possible.

Accordingly, the inventor proposes an improvement of the method for the reconstruction of sectional images from detector measurement data of a tomography device, at least one radiation source which is moved about a system axis, and at least one detector D lying opposite and having at least one detector row and in each case a multiplicity of detector elements, which measures the absorption of the radiation emerging from the radiation source after penetrating an examination object. At least the radiation source with its focus, preferably also the detector D, rotates on an imaginary cylinder, preferably a multiplicity of circular paths or a spiral path, around the examination object. Further, a system axis as axis of rotation, in the process scanning said examination object—lying in a scanning volume formed by the beams Rₛ—by way of the beams Rₛ and, per detector row, records a sinogram A including a multiplicity of partial sinograms Aₛ corresponding to the number of detector elements which are respectively assigned to a detector element Dₛ of a detector row.

An improvement of at least one embodiment includes a calculation that follows the following specification:

the scanning volume is divided into a multiplicity of partial volumes,

for each partial volume a reference beam is sought, which intersects said partial volume and which is at the greatest distance from the system axis, and

the absorption coefficients of each partial volume are calculated exclusively with absorption values that originate from beams whose distance is greater than or equal to the distance between the reference beam and the system axis.

In principle, the form of the partial volumes is insignificant in the case of this embodiment. By way of example, rectangular volumes arranged in checkered fashion or partial volumes that are hexagonal in projection or other volume forms may also be assumed. On account of this computation specification that is oriented to the actual geometrical situation of a rotation fan beam, the computational complexity is substantially reduced and an explicit calculation of the adsorption coefficients now becomes possible even at high resolution.

Preferably, the sum of all partial volumes forms an overall volume that is formed convexly—relative to the system axis—, where convex in the sense of this document is to be regarded as a scanning volume which is divided into partial volumes and curved inward toward the system axis. In this case, all connecting lines between the partial volumes are permitted to intersect only other partial volumes or partial volumes which do not contain an examination object.

If it is desired to choose a particularly favorable geometry which results from the method of operation and arrangement of a CT, then the scanning volume can be divided into a multiplicity of shells Sₛ and each shell can in turn be divided into a multiplicity of shell elements Sₛᵢ. Further, the absorption coefficients of each shell element Sₛᵢ can be calculated, each shell Sₛ being defined by a rotating beam Rₛ between focus and detector element Dₛ of the detector D which intersects said shell as most centrally located shell. Per shell Sₛ, the absorption coefficients μₛᵢ of each shell segment are formed as a function of the partial sinograms Aₛ of the beam-defining detector element Dₛ and the partial sinograms Aₓ x:=1 to s-1, which originate from beams Rₓ lying further outward in the beam fan.

According to an embodiment of the invention, the absorption coefficient μₛᵢ of the i-th shell segment Sₛᵢ of the s-th shell Sₛ can be calculated by use of the following formula:

$${μ}_{si} = {\sum }_{x}^{s} {\sum }_{y}^{p} {C}^{( s , i )} {xyA}_{xy}$$

Where C^{(s,i)}_{xy} is the shell coefficient matrix of the s-th shell and the i-th segment and A_{xy} is the sinogram and the summation proceeds over all projections y=1 . . . p and the shells i=1 . . . s.

The shell coefficient matrix C^{(s,i)}_{xy} is a function of the variables x=1 . . . s and y=1 . . . p. It is defined by way of the path length matrices as

$$\begin{matrix}{C}^{( s , i )} = - {L}_{s}^{- 1} {\sum }_{i = 1}^{s - 1} {L}_{s , i} {\sum }_{j = 1}^{i} {C}_{i , j} δ ( j , n ) \\ ⇒ {C}^{( s , i )} = - {L}_{s}^{- 1} {\sum }_{i = n}^{s - 1} {L}_{s , i} {C}_{i , n}\end{matrix}$$

This produces for example:
C^{(1,1)}=L₁⁻¹
C^{(2,2)}=L₂⁻¹ C^{(2,1)}=−L₂⁻¹L_{2,1}L₁⁻¹
C^{(3,3)}=L₃⁻¹ C^{(3,2)}=−L₃⁻¹L_{3,2}L₂⁻¹ C^{(3,1)}=L₃⁻¹L_{3,2}L₂⁻¹L_{2,1}L₁⁻¹−L₃⁻¹L_{3,1}L₁⁻¹

The shell coefficient matrix is thus to be established for each shell Sₛ and each segment sₛᵢ. Its size increases as the shell index increases. It has the dimension [1, 1 . . . p], for the first shell, [1 . . . 2, 1 . . . p] for the second shell etc.

The dependence on the shell segment is trivial in the advantageous rotationally symmetrical case: the matrix C^{(s,i+1)} is produced from C^{(s,i)} by permutation of the entire matrix by one place.

The application of this mode of calculation permits the situation that the matrix elements are already calculated prior to the actual evaluation of the measurement results and, as previously calculated constants, only have to be multiplied by the associated absorption values during the evaluation.

It is advantageous in the application of an embodiment of this method if the individual shells have an identical thickness and the shell segments thus also have an identical thickness in the radial direction. Furthermore, the individual shell segments may have an identical length in the circumferential direction and/or an identical cross-sectional area perpendicular to the system axis.

Dividing the shells into shell segments with segment angles of identical magnitude is particularly favorable. Furthermore, the examination volume may be divided such that each beam of the beam bundle describes a tangent circle with all perpendicular reference points with respect to the system axis, and the shell segments, which have an outer circle segment and an inner circle segment, the shell segments being arranged in such a way that the tangent circle lies centrally between outer circle segment and inner circle segment.

In the case of scanning by way of a spiral course of the radiation source, it is particularly advantageous if the shell segments have an imaginary centroid line corresponding to segments of concentrically arranged helical lines about the system axis.

Furthermore, with the method according to an embodiment of the invention, the energy dependence of the absorption coefficients can also be taken into account in the measurement of the coefficients. The energy-dependent intensity alteration of the radiation after passage through the examination object may be observed for this purpose. For example, the total intensity alteration of at least two beams with a known different energy spectrum on the same beam path may be used. This may be done, for example, for the purpose of scanning at least two, preferably exactly two or three, radiation sources with different energy spectra, preferably with in each case a detector lying opposite. They may be, for example, arranged in such a way that they rotate around the examination object on the same path during the scanning.

On the other hand, the examination object may also be scanned with a beam bundle with a known energy spectrum and the altered energy spectrum of each beam after passage through the examination object is measured, in which case the energy spectrum may advantageously comprise two or three average energies.

For the representation of the CT sectional images, an intensity value of a primary color (RGB, YMC) may in this case be assigned to the value of the energy-dependent absorption coefficients per energy, which results in a color representation of the CT image.

In accordance with a concept of an embodiment of the invention, the inventor also proposes the improvement of a tomography device, preferably CT device, for the reconstruction of sectional images from detector measurement data. In this case, this known tomography device has at least one radiation source which can be moved about a system axis, and at least one at least single-row detector lying opposite, which measures the absorption of the radiation emerging from the radiation source after penetrating an examination object. At least the radiation source, preferably also the detector rotating on an imaginary cylinder surface, preferably on a multiplicity of circular paths or a spiral path, moves around the examination object and in the process scans the examination object—lying in a scanning volume formed by the beams—by way of beam bundles.

A device, for example at least one computing unit, may be used for the control of the tomography device, and also collection and computational processing of detector output data, reconstruction of tomographic images and representation of the images. According to an embodiment of the invention, the computing unit may be equipped with program segments for carrying out the methods outlined above and process at least parts of the method according to an embodiment of the invention during operation of the computing unit.

### BRIEF DESCRIPTION OF THE DRAWINGS

The invention is described in more detail below on the basis of example embodiments with the aid of the figures, attention being drawn to the fact that only the elements that are essential for a direct understanding of the invention are shown. In this case, the following reference symbols are used: 1: computer tomography device; 2: X-ray tube; 3: detector; 4: patient's couch; 5: system axis; 6: gantry; 7: patient; 8: memory; 9: data/control line; 10: computing unit; 11: screen; 12: keyboard; 13: focus; 14: excerpt; 15: scanning volume; 16: rectangular partial volume; 17: hexagonal partial volume; A: sinogram; Aₛ: partial sinograms; D: detector; Dₛ: detector elements; lₛ: passage lengths of the beams through shell segments; Lₛᵢ: matrices; P₁-Pₙ: computer programs; Rₛ: X-ray beams; Sₛ: shells; sₛᵢ: shell segments; φ: angle of rotation of the X-ray tube; μₛᵢ: absorption coefficients; Δφ segment angle.

Specifically:

FIG. 1 illustrates a CT device with a computing unit;

FIG. 2 illustrates a section through the beam path with shell-like division of the scanning volume;

FIG. 3 illustrates a sinogram;

FIG. 4 illustrates a partial sinogram;

FIG. 5 illustrates the illustration from FIG. 2 plus two angularly offset beam courses;

FIG. 6 illustrates an excerpt from FIG. 5;

FIG. 7 illustrates checkered partial volumes;

FIG. 8 illustrates partial volumes arranged in honeycomb fashion;

FIG. 9 illustrates a 3-D illustration of a shell-like division of the scanning volume in the case of multi-row detectors.

### DETAILED DESCRIPTION OF THE EXAMPLE EMBODIMENTS

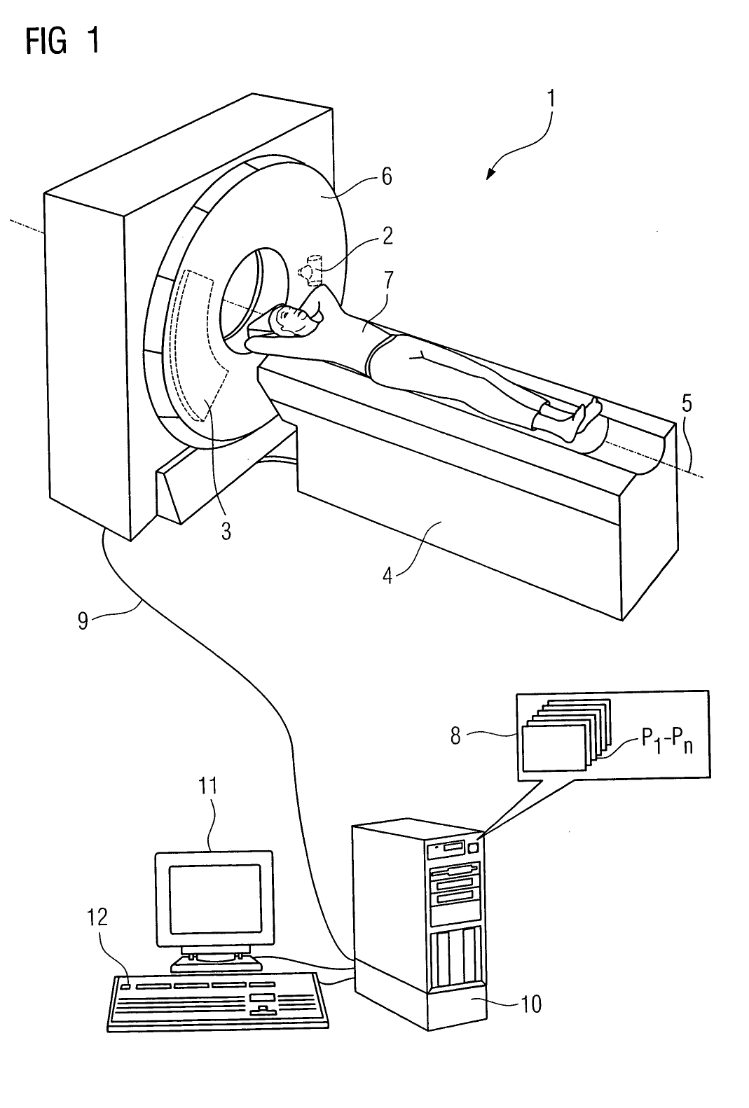

A computer tomography device 1 known per se—such as is illustrated in FIG. 1—may be used for implementing the method according to an embodiment of the invention. Such a computer tomography device has at least one X-ray tube with at least one focus which generates a beam bundle that impinges on a detector 3 lying opposite. In the embodiment of the computer tomography device that is shown here, the X-ray tube 2 and the detector 3 move on a gantry 6 circularly around an examination object—here a patient 7—and in this case scan the examination object with its X-ray beams. The absorption of the X-ray beams is measured in the detector 3 by a multiplicity of detector elements, conducted via a data and control line 9 to a computing unit 10 and stored and processed there.

For operating the computing unit 10 and thus also the computer tomograph 1, the computing unit has a screen 11 and an input unit in the form of a keyboard 12, by which the computer tomograms determined can be both controlled and output. The actual calculation method takes place in the computing unit 10, which has a memory—represented symbolically here by the reference symbol 8—in which not only the data but also the execution programs P₁-Pₙ are stored.

In accordance with the method according to an embodiment of the invention, the examination object 7 can be scanned spirally by the patient's couch 4 being advanced continuously along the system axis 5. A simpler variant consists in carrying out the advance sequentially, so that an advance takes place after every 360° scanning and the actual scanning is performed when the examination object 7 is in a rest state. Both variants can be carried out by way of the method according to an embodiment of the invention.

It is additionally pointed out that both single-row and multiple-row detectors can be used. It is possible to utilize one or a plurality of X-ray tubes with in each case one or a plurality of foci with in turn a single or a plurality of moving or stationary detectors. All that is essential to the method is that a beam bundle formed in fan-type fashion scans the examination object in a rotational movement about the system axis.

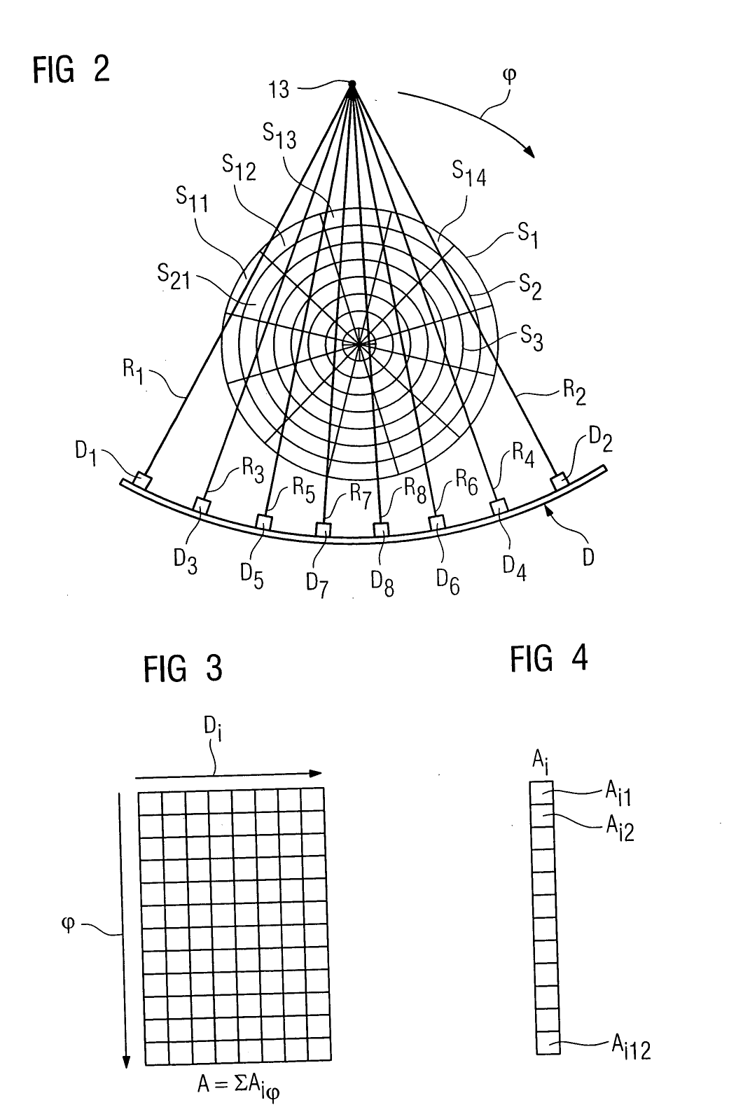

A special variant of the calculation is illustrated in FIGS. 2 to 6. This variant relates to the scanning volume being divided into segments arranged in shell-like fashion, as is illustrated in FIG. 2.

This FIG. 2 shows a focus 13, from which a fan-type beam bundle with individual X-ray beams R₁ to R₈ impinges on a detector D lying opposite and having detector elements D₁ to D₈. During the rotation of the focus and detector about the system axis, each beam R₁ to R₈ scans a shell volume S₁ to S₈, which is in turn divided into 12 shell segments Sₛᵢ in accordance with the number of measurement points considered. Consequently, a single shell is assigned to each X-ray beam, the detector elements associated with the X-ray beams being counted in accordance with their distance from the central axis. In the illustration shown, which also corresponds to most detectors used, the detector elements are arranged on the detector in a manner slightly offset with respect to the center, so that no redundancies arise between the measurement values of the detectors.

If the results of the scanning, that is to say the detector output values, are plotted for each beam or each detector element and each rotational angle φ, then a sinogram A such as is illustrated in FIG. 3 is obtained, each column corresponding to the measurement points of a detector element and each row being assigned to the individual measurement points during a 360° rotation.

If an individual partial sinogram is considered, that is to say the measurement values of an individual detector element for a full rotation, then the partial sinogram such as is shown in FIG. 4 is obtained, which in this case likewise has 12 individual values Aᵢ₁ to Aᵢ₁₂ corresponding to the number of shell segments of a shell. Each individual value of this partial sinogram corresponds to the absorption of an X-ray beam that impinges on this detector element under consideration, at the corresponding measurement angle.

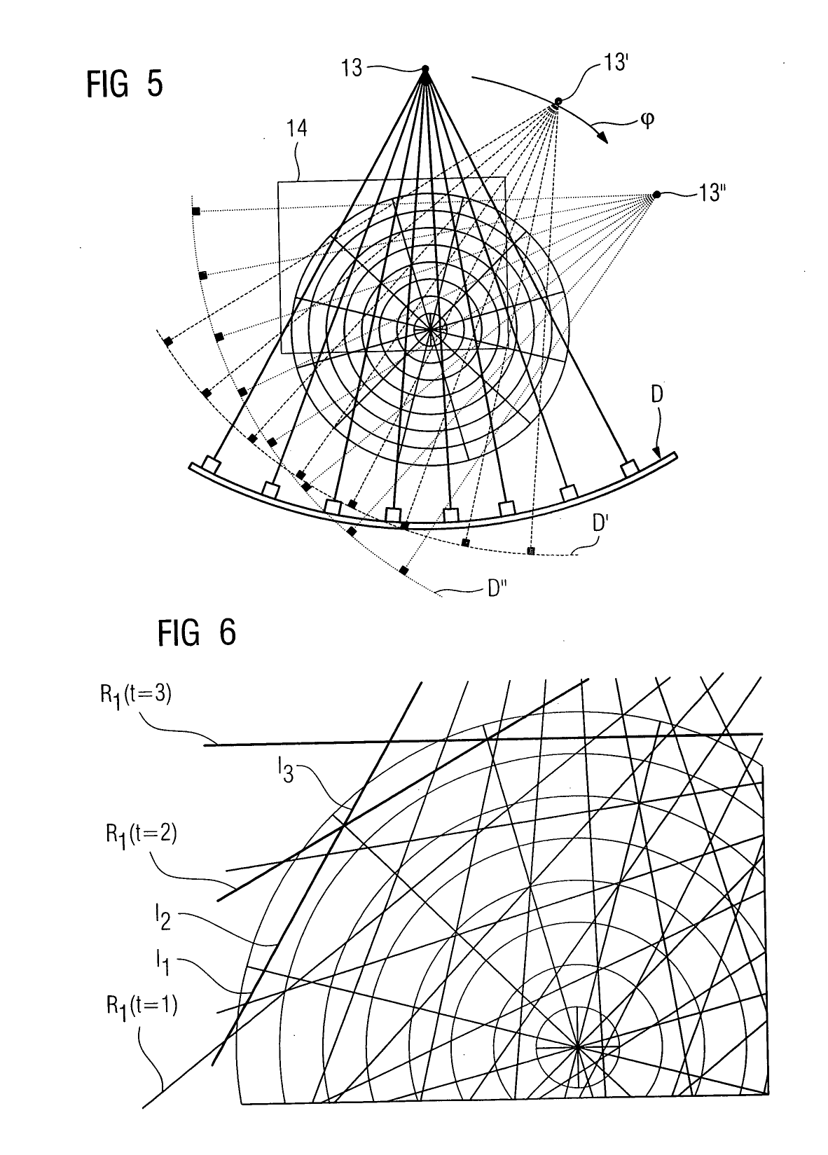

FIG. 5 illustrates the scanning process of the beam fan at three different angles φ of rotation. The rotated situations are identified by one ′ or two ′. During each partial rotation, the X-ray beams are displaced to an extent such that a new shell segment is penetrated in their center, further other shell segments likewise being touched in the edge zones. It should be noted, in particular, that during a 360° rotation, exclusively segments of the outer shell S₁ are penetrated by the outer X-ray beam R₁. As such, a calculation of the absorption coefficients of the outer shell without taking account of the values of different detector elements than of the detector element D₁ is possible and can thus be carried out in a very simple manner computationally.

If the nearest X-ray beam R₂ location further inward is considered, then the latter penetrates only the outer shell and the second shell. Thus, only the measurement values of the detector elements D₁ and D₂ are required for calculating the absorption coefficients of the shell segments of the second shell. This mode of consideration can be carried out up to the inner shell, so that this results in a very simplified explicit calculation of the absorption coefficients.

FIG. 6 shows the paths of the X-ray beams through the scanning volume subdivided in shell-like fashion once again in an enlarged illustration of the excerpt 14 from FIG. 5. The X-ray beam R₁ (t=1, 2 and 3) is emphasized. It can readily be discerned that the X-ray beam R, (t=1) with the length L₂ penetrates the shell segment assigned to it on its greatest length, while the marginal lengths L₁ and L₃ relate to the adjacent beam segments.

The X-ray beam R₁ moves through the scanning volume in accordance with the rotation of the focus and detector. The effective path lengths with which the individual shell segments are penetrated are readily calculable so that a simple calculation of the absorption coefficients of the individual shell segments is possible in a corresponding prepared matrix.

FIGS. 2 to 6 illustrate the division of the scanning volume into individual partial volumes arranged in shell-type fashion. The calculation is particularly simple on account of its geometrical association with the scanning method of a cyclically rotating focus. However, the method according to an embodiment of the invention is in no way restricted to such a shell-like arrangement of scanning volumes, rather it may, for the person skilled in the art, equally be applied to other subdivisions of the scanning volume.

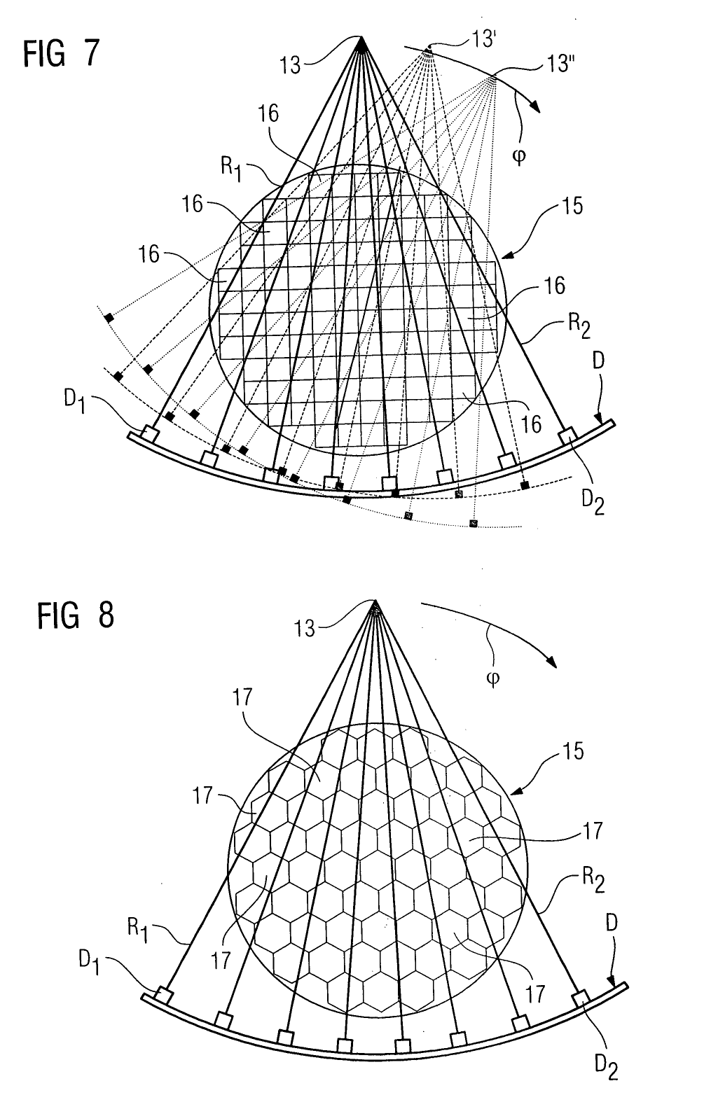

By way of example, FIG. 7 illustrates a division of the scanning volume 15 into a multiplicity of partial volumes 16 with a square cross section. In addition, the scanning of the scanning volume by a beam fan—emerging from a focus 13—with X-ray beams Rₓ at different angles φ of rotation is indicated by the appending of ′ and ″. The basic principle of the calculation as outlined above is basically preserved.

In this case, it is necessary merely to take into consideration the fact that, in the calculation of the absorption coefficients of the partial volumes 16, firstly the outer partial volumes which are penetrated by an outer first beam are taken into account and then the calculation is continued progressively to partial volumes 16 which lie further inward and are permeated by the respective nearest inner X-ray beams. A corresponding simplification of the computation as a consequence is discernable in this case, too, as a result of which an explicit calculation at high resolution is made possible with this division of the scanning volume 15, too.

In addition, FIG. 8 shows a division of the scanning volume 15 into partial volumes 17 with a hexagonal cross section, in which case the computation method according to an embodiment of the invention can be employed for a division of this type as well.

The examples outlined previously in each case involve the consideration of a focus/detector combination with a single detector that rotates on a circular path around an examination object. According to an embodiment of the invention, it is also possible to apply the method described to such a focus/single-row doctor combination which moves spirally, that is to say with a simultaneous advance relative to the examination object, so that a spiral path is scanned. Correspondingly, it is also possible to extend the method to a detector having a plurality or multiplicity of rows, in which case the detector can move both on a circular path and on a spiral path.

An embodiment of the inventive method, the shell reconstruction, has hitherto been described as a pure 2D layer method. In principle, this 2D method can also be applied to multi-row detectors in a simple manner if the angular expansion of the beam bundle is disregarded in an idealized manner. The text below will describe taking account of the fan angle for static CT reconstruction and finally also the conversion for spiral CT data.

For the conversion of the shell reconstruction in 3D, first a transition is made from a reconstruction in polar coordinates to a reconstruction in cylinder coordinates. The system axis is thus obtained as a third spatial coordinate. The sections of the beam lines with the 2D shells becomes the section with a cylinder.

For multi-row CT systems and in particular area detectors, the fan angle between the tube-detector center axis and the z position (z axis=system axis) of a detector row may be more than 10 degrees. In the case of static rotation of detector and tube, a plane is no longer defined for fan angle γ>0. Instead, a “diablo”- or “saucer”-shaped volume results during a rotation.

The individual rows of the detector are reconstructed independently of one another according to the invention. In this case, the “sectional shells” of a detector row have an inclination γ relative to the local center perpendicular between tube and detector. The 360° rotation of the center perpendicular about the patient axis gives rise to a diablo-shaped sectional volume that is to be handled by means of the shell reconstruction. For this purpose, the shells are established for the plane where γ=0. The absorption lengths of the beams through the shell segments considered rise by a factor of 1/cos(γ)>1 as a result of the fan angle. Otherwise, the reconstruction is carried out as described above for each rotation for p projections.

Thus, in the 2D reconstruction method of an embodiment described above, all that is produced is a change in the matrices Lₛ and Lₛᵢ which are scaled with a factor of 1/cos(y). As a result, the method then reconstructs a “diablo” instead of a plane. The conditioning of these data in system-axis layer planes can now be carried out as follows.

Center-plane approximation: if planes in the center plane of the “diablos” are to be reconstructed, then the extended form can be disregarded for small fan angles: this is also due to the fact that, for the regions of the outer shells that are extended in the z direction, contributions from volumes on both sides of the plane are introduced in uniformly weighted fashion. The method directly supplies as a result the reconstruction of nₘₐₓ (=number of detector rows) center planes. This information can also be e.g. linearly interpolated to other z planes.

Radius-dependent Z interpolation: the dependence of the layer resolution on the radius is primarily a property of the CT multi-row measurement method. In order to obtain a homogeneous solution, if appropriate, for larger fan angles and for the 3D spiral, the following is taken into consideration: firstly, the center planes are calculated as described above by means of the shell reconstruction for each detector row. Afterward, the resulting “diablo” volumes are mapped onto arbitrary intervening z planes with compensation of the radius-dependent resolution.

For the filtering, there is the aim here of achieving a uniformly filtered z extent in the projected z plane. The linear extent in the z direction of the “diablo” as a function of the shell radius r with 2r*tan(γ) and the pixel aperture of the detector are to be taken into consideration for this purpose. In a similar manner to that in the 2D method, over and above the homogenization of the plane no low-pass filtering is pursued for noise reduction. Thus in 3D as well, the maximum information is to be extracted from the raw data and the desired noise/sharpness value is to be set only in the representation.

For the shell reconstruction of a spiral scanning, what is chosen in principle is exactly the same procedure as in the reconstruction taking account of the fan angle. The volume of a 360 degree rotation that is defined by the elliptical sectional plane is now dependent on the size of the CT advance. In the case of a very fast advance there are two inclined elliptical planes as boundary of the cylinder section. The resulting volume has the appearance, as it were, of an obliquely cut slice of sausage. In the case of small table advance, by contrast, one ends up with the diablo again. Average advance values around 1 finally produce transitions between the two forms.

Precisely as in the case of taking account of the fan angle alone, 360 degree rotation data are now reconstructed with respect to this volume and afterward, as described above, converted to arbitrary interpolating z planes.

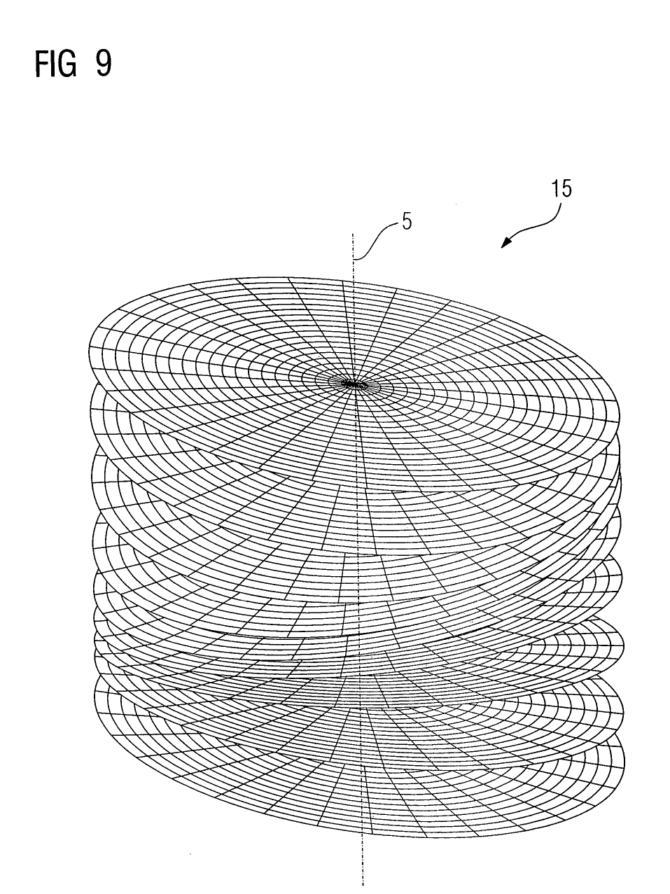

An example 3D scanning of a scanning volume 15 and the division thereof into shell segments arranged in shell-shaped fashion is shown in FIG. 9.

Overall, then, embodiments of the invention describes a very simple reconstruction method that is increased or even optimized in respect of computational time, in which method the scanning volume is divided into a multiplicity of partial volumes, for each partial volume a reference beam is sought, which intersects said partial volume and which is at the greatest distance from the system axis, and the absorption coefficients of each partial volume are calculated exclusively with absorption values that originate from beams whose distance is greater than or equal to the distance between the reference beam and the system axis.

It goes without saying that the abovementioned features of embodiments of the invention can be used not only in the combination respectively specified, but also in other combinations or by themselves, without departing from the scope of the invention. Any of the aforementioned methods may be embodied in the form of a system or device, including, but not limited to, any of the structure for performing the methodology illustrated in the drawings.

Further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, such as floppy disks (trademark), cassette tapes, and removable hard disks; media with a built-in rewriteable non-volatile memory, such as memory cards; and media with a built-in ROM, such as ROM cassettes.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for use in the reconstruction of sectional images from detector measurement data of a tomography device, the device including,
at least one radiation source, movable about a system axis, and
at least one detector lying opposite and having at least one detector row and in each case a multiplicity of detector elements, to measure the absorption of the radiation emerging from the radiation source after penetrating an examination object, at least the radiation source with its focus being rotatable on an imaginary cylinder around the examination object and a system axis as axis of rotation, in the process scanning the examination object, lying in a scanning volume formed by the beams, via the beams and, per detector row, recording a sinogram including a multiplicity of partial sinograms corresponding to the number of detector elements which are respectively assigned to a detector element of a detector row, the method comprising:
dividing the scanning beam into a multiplicity of partial volumes;
determining a reference volume, for each partial volume, which intersects the partial volume and which is at the greatest distance from the system axis, and
calculating the absorption coefficients of each partial volume exclusively with absorption values that originate from beams whose distance is greater than or equal to the distance between the reference beam and the system axis.

2. The method as claimed in claim 1, wherein the scanning volume is divided into a multiplicity of shells and each shell is in turn divided into a multiplicity of shell elements, and wherein the absorption coefficients of each shell element are calculated such that each shell is defined by a rotating beam between focus and detector element of the detector which intersects the shell as most centrally located shell, and per shell the absorption coefficients of each shell segment are formed as a function of the partial sinograms of the beam-defining detector element and the partial sinograms, which originate from beams lying further outward in the beam fan.

3. The method as claimed in claim 2, wherein the absorption coefficient of the i-th shell segment of the s-th shell is calculated by way of the following formula:
$${μ}_{si} = {\sum }_{x}^{s} {\sum }_{y}^{p} {C}^{( s , i )} {xyA}_{xy}$$
where C^{(s,i)}_{xy} is the shell coefficient matrix of the s-th shell and the i-th segment and A_{xy} is the sinogram and the summation proceeds over all projections y=1 . . . p and the shells i=1 . . . s.

4. The method as claimed in claim 1, wherein the sum of all partial volumes forms a convexly formed overall volume.

5. The method as claimed in claim 1, wherein at least one of the partial volumes and individual shell segments have an identical thickness in the radial direction.

6. The method as claimed in claim 1, wherein at least one of the partial volumes and individual shell segments have an identical arc length in the circumferential direction.

7. The method as claimed in claim 1, wherein at least one of the partial volumes and individual shell segments have an identical cross-sectional area perpendicular to the system axis.

8. The method as claimed in claim 1, wherein at least one of the partial volumes and individual shell segments sweep over a segment angle of identical magnitude.

9. The method as claimed in claim 2, wherein a shell with constant distance from the system axis is assigned to each beam proceeding from the focus with respect to a specific detector element.

10. The method as claimed in claim 1, wherein each beam of the beam bundle describes a tangent circle with all perpendicular reference points with respect to the system axis, and the shell segments, which have an outer circle segment and an inner circle segment, the shell segments being arranged in such a way that the tangent circle lies centrally between outer circle segment and inner circle segment.

11. The method as claimed in claim 3, wherein at least one of the partial volumes and shell segments (sₛᵢ) are formed helically about the system axis.

12. The method as claimed in claim 3, wherein at least one of the partial volumes and shell segments (sₛᵢ), the further away from the system axis they are and the larger their angle between beam and system axis, have a larger extent in this axial direction.

13. The method as claimed in claim 1, wherein the energy dependence of the absorption coefficients is taken into account in the measurement of said coefficients.

14. The method as claimed in claim 13, wherein the energy-dependent intensity alteration of the radiation after passage through the examination object is used for this purpose.

15. The method as claimed in claim 13, wherein the total intensity alteration of at least two beams with a known different energy spectrum on the same beam path is used for this purpose.

16. The method as claimed in claim 15, wherein at least two radiation sources with different energy spectra are used, which are arranged in such a way that they rotate around the examination object on the same path during the scanning.

17. The method as claimed in claim 15, wherein the examination object is scanned with a beam bundle with a known energy spectrum and the altered energy spectrum of each beam after passage through the examination object is measured.

18. The method as claimed in claim 17, wherein the energy spectrum comprises at least two average energies.

19. The method as claimed in claim 15, wherein, in the representation of the CT sectional images, an intensity value of a primary color is assigned to the value of the energy-dependent absorption coefficients per energy, which results in a color representation of the CT image.

20. A tomography device, for use in the reconstruction of sectional images from detector measurement data, comprising:
at least one radiation source, movable about a system axis;
at least one at least single-row detector lying opposite, to measure the absorption of the radiation emerging from the radiation source after penetrating an examination object, the at least the radiation source rotating on an imaginary cylinder surface around the examination object and in the process scanning said examination object, lying in a scanning volume formed by the beams, via beam bundles; and
means for controlling the tomography device, and for collecting and computational processing detector output data, for reconstruction of tomographic images and representation of the images, and for carrying out the method as claimed in claim 1.

21. The method as claimed in claim 3, wherein the sum of all partial volumes (sₛᵢ) forms a convexly formed overall volume.

22. The method as claimed in claim 3, wherein at least one of the partial volumes and individual shell segments (sₛᵢ) have an identical thickness in the radial direction.

23. The method as claimed in claim 3, wherein at least one of the partial volumes and individual shell segments (sₛᵢ) have an identical arc length in the circumferential direction.

24. The method as claimed in claim 3, wherein at least one of the partial volumes and individual shell segments (sₛᵢ) have an identical cross-sectional area perpendicular to the system axis.

25. The method as claimed in claim 3, wherein at least one of the partial volumes and individual shell segments (sₛᵢ) sweep over a segment angle (Δφ) of identical magnitude.

26. The method as claimed in claim 14, wherein the total intensity alteration of at least two beams with a known different energy spectrum on the same beam path is used for this purpose.
